SEQUENCE LISTING (15 sequences)

SEQ ID NO: 1 - DNA, 19 bp, Artificial Sequence
  Synthetic oligonucleotide
ggagaaaagggacagaagc

SEQ ID NO: 2 - DNA, 18 bp, Artificial Sequence
  Synthetic oligonucleotide
acgacagctacacttagt

SEQ ID NO: 3 - DNA, 1362 bp, Ovis aries
atggcgcttcccaacaaattcttcctttggttttgctgctttgcctggctctgttttcctattagccttgattctctgccttctaggggagaagctcagattgtagctaggactgcgttggaatctgaggctgagacttggtccttgctgaaccatttaggtgggagacacagacctggtctcctttcccctctcttagaggttctgtatgatgggcacggggaaccccccaggctgcagccagatgacagagctttgcgctacatgaagaggctctataaggcatacgctaccaaggaggggacccctaaatccaacagacgccacctctacaacactgttcggctcttcaccccctgtgctcagcacaagcaggctcctggggacctggcggcaggaacctttccatcagtggatctgctgtttaacctggatcgtgttactgttgtggaacatttattcaagtcagtcttgctgtatactttcaacaactccatttcttttccctttcctgttaaatgtatatgcaacctggtgataaaagagccagagttttctagcaagactctccctagagctccatactcatttacctataactcacagtttgaatttagaaagaaatacaaatggatggagattgatgtgacggctcctcttgagcctctggtggcctcccacaagaggaatattcacatgtctgtaaattttacatgtgcggaagaccagctgcagcatccttcagcgcgggacagcctgtttaacatgactcttctcgtagcgccctcactgcttttgtatctgaacgacacaagtgctcaggcttttcacaggtggcattccctccaccctaaaaggaagccttcacagggtcctgaccagaagagagggctatctgcctaccccgtgggagaagaagctgctgagggtgtaagatcgtcccgtcaccgcagagaccaggagagtgccagctctgaattgaagaagcctctggttccagcttcagtcaatctgagtgaatacttcaaacagtgtctttttccccagaatgaatgtgagctccatgactttagacttagctttagtcagctgaagtgggacaactggattgtggccccacacaaatacaaccctcgatactgtaaaggggactgtcccagggcggtcggacatcggtatggctctccggttcacaccatggtgcagaacatcatccatgagaaacttgactcctcagtgccaagaccatcctgtgtacctgccaagtatagccctttgagtgttttggccatcgagcctgatggctcaatcgcttataaagaatatgaagatatgatagccactaagtgtacctgtcgttaa

SEQ ID NO: 4 - protein, 453 aa, Ovis aries
MALPNKFFLWFCCFAWLCFPISLDSLPSRGEAQIVARTALESEAETWSLLNHLGGRHRPGLLSPLLEVLYDGHGEPPRLQPDDRALRYMKRLYKAYATKEGTPKSNRRHLYNTVRLFTPCAQHKQAPGDLAAGTFPSVDLLFNLDRVTVVEHLFKSVLLYTFNNSISFPFPVKCICNLVIKEPEFSSKTLPRAPYSFTYNSQFEFRKKYKWMEIDVTAPLEPLVASHKRNIHMSVNFTCAEDQLQHPSARDSLFNMTLLVAPSLLLYLNDTSAQAFHRWHSLHPKRKPSQGPDQKRGLSAYPVGEEAAEGVRSSRHRRDQESASSELKKPLVPASVNLSEYFKQCLFPQNECELHDFRLSFSQLKWDNWIVAPHKYNPRYCKGDCPRAVGHRYGSPVHTMVQNIIHEKLDSSVPRPSCVPAKYSPLSVLAIEPDGSIAYKEYEDMIATKCTCR

SEQ ID NO: 5 - protein, 135 aa, Homo sapiens
GQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 6 - protein, 135 aa, Pan troglodytes
GQETVSSELKKPLGPASFNLSEYFKQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 7 - protein, 135 aa, Ovis aries
DQESASSELKKPLVPASVNLSEYFKQFLFPQNECELHDFRLSFSQLKWDNWIVAPHKYNPRYCKGDCPRAVGHRYGSPVHTMVQNIIHEKLDSSVPRPSCVPAKYSPLSVLAIEPDGSIAYKEYEDMIATKCTCR

SEQ ID NO: 8 - protein, 135 aa, Capra hircus
DQESVSSELKKPLVPASVNLSEYFKQFLFPQNECELHDFRLSFSQLKWDNWIVAPHKYNPRYCKGDCPRAVGHRYGSPVHTMVQNIIHEKLDSSVPRPSCVPAKYSPLSVLAIEPDGSIAYKEYEDMIATKCTCR

SEQ ID NO: 9 - protein, 135 aa, Bos taurus
DQESVSSELKKPLVPASFNLSEYFKQFLFPQNECELHDFRLSFSQLKWDNWIVAPHKYNPRYCKGDCPRAVGHRYGSPVHTMVMNIIHEKLDSSVPRPSCVPAKYSPLSVLAIEPDGSIAYKEYEDMIATKCTCR

SEQ ID NO: 10 - protein, 135 aa, Sus scrofa
AQDTVSSELKKPLVPASFNLSEYFKQFLFPQNECELHDFRLSFSQLKWDNWIVAPHKYNPRYCKGDCPRAVGHRYGSPVHTMVQNIIHEKLDSSVPRPSCVPAKYSPLSVLAIEPDGSIAYKEYEDMIATKCTCR

SEQ ID NO: 11 - protein, 135 aa, Canis lupus
GQDTVSLELHKPLAPASFNLSEYLKHFLFPQHECELHDFRLSFSQLKWDNWIVAPHRYNPRYCKGDCPRAVGHRYGSPVHTMVQNIIHEKLNSSVPRPSCVPAKYSPLSVLTIEPDGSIAYKEYEDMIATKCTCR

SEQ ID NO: 12 - protein, 135 aa, Mus musculus
GQKAIRSEAKGPLLTASFNLSEYFKQFLFPQNECELHDFRLSFSQLKWDNWIVAPHRYNPRYCKGDCPRAVRHRYGSPVHTMVQNIIYEKLDPSVPRPSCVPGKYSPLSVLTIEPDGSIAYKEYEDMIATRCTCR

SEQ ID NO: 13 - protein, 134 aa, Rattus rattus
GQKTLSSETKKPLTASFNLSEYFRQFLFPQNECELHDFRLSFSQLKWDNWIVAPHRYNPRYCKGDCPRAVRHRYGSPVHTMVQNIIYEKLDPSVPRPSCVPGKYSPLSVLTIEPDGSIAYKEYEDMIATRCTCR

SEQ ID NO: 14 - protein, 135 aa, Ovis aries
DQESASSELKKPLVPASVNLSEYFKQCLFPQNECELHDFRLSFSQLKWDNWIVAPHKYNPRYCKGDCPRAVGHRYGSPVHTMVQNIIHEKLDSSVPRPSCVPAKYSPLSVLAIEPDGSIAYKEYEDMIATKCTCR

SEQ ID NO: 15 - DNA, 18 bp, Artificial Sequence
  Synthetic oligonucleotide
gaccaggagagtgtcagc

(19) United States
(12) Patent Application Publication
(10) Pub. No.: US 2011/0252489 A1
(43) Pub. Date: Oct. 13, 2011

(54) METHOD OF IDENTIFYING PROLIFICACY IN MAMMALS

(75) Inventors: Eduardo de Oliveira Melo, Brasilia-DF (BR); Samuel Rezende Paiva, Brasilia-DF (BR); Carlos Jose Hoff de Souza, Bage-RS (BR)

(73) Assignee: EMPRESA BRASILEIRA DE PESQUISA AGROPECUARIA-EMBRAP, BRASILIA-DF (BR)

(21) Appl. No.: 12/744,288

(22) PCT Filed: Nov. 24, 2008
(86) PCT No.: PCT/BR08/00356

§ 371 Date: Nov. 3, 2010

(30) Foreign Application Priority Data
Nov. 23, 2007 (BR) PI0706173-0

(51) Int. Cl. C12Q 1/68; C07K 14/435; A01K 67/027; C12N 15/85; C12N 5/10; G01N 27/26; C12N 15/12

(52) U.S. Cl. 800/13; 204/450; 204/456; 530/350; 536/23.5; 435/320.1; 435/328; 435/6.11; 435/6.12

(57) ABSTRACT

The present invention refers to a method of predicting prolificacy in mammals, by means of analyzing a specific molecular marker for a novel mutation correlated to the increase of the ovulation rate. The correct and simple identification of the presence of the mutation, provided by the use of the method can be highly useful in the genetic improvement of ewes, as well as flock reproduction management. The invention also refers to a novel mutation in the GDF-9 gene which positively alters the ovulation rate and to the use of said genetic sequence for the production of high prolificacy transgenic animals.

### FIELD OF THE INVENTION

The present invention refers to a method of predicting prolificacy in mammals, by means of analyzing a specific molecular marker for a novel mutation correlated to the increase of the ovulation rate. The correct and simple identification of the presence of the mutation, provided by the use of the method can be highly useful in the genetic improvement of ewes, as well as flock reproduction management. The invention also refers to a novel mutation in the GDF-9 gene which positively alters the ovulation rate and to the use of said genetic sequence for the production of high prolificacy transgenic animals.

### STATE OF THE ART

The “Growth Differentiation Factors” (GDFs) are peptide hormones that form the largest sub-group of growth factors of the TGF-β superfamily of regulatory polypeptides. The hormone GDF-9 is produced by the ovocyte and plays a fundamental role in fertility, given its working in folliculogenesis and follicular development, stimulating growth and differentiating the somatic cells from the ovarian follicles. The GDF-9 gene, situated in chromosome 5 in sheep, is constituted of two exons, and the second contains over 70% of the sequence of protein reading frames and encodes, in its C-terminal portion, a mature peptide with 135 aminoacids (Hanrahan, J. P., Gregan, S. M., Mulsant, P., Mullen, M., Davis, G. H., Powell, R. and Galloway, S. M. Mutations in the genes for oocyte-derived growth factors GDF-9 and BMP-15 are associated with both increased ovulation rate and sterility in Cambridge and Belclare ewes (Ovis aries). Biol Reprod, 70, 900-909. 2004). The hormone GDF-9 is formed of 2 dimers of this peptide and its tertiary structure is maintained by a “Knot” of 6 highly conserved cysteines in the TGF-β family (Vitt, U A., Hsu, S. Y. and Hsueh, A. J. W. Evolution and classification of cysteine knot-containing hormones and related extracellular signaling molecules. Mol Endocrinol, 15(5), 681-694.2001).

Synthesized and secreted by the ovocyte since the beginning of the primary follicle phase, the GDF-9 is present in the follicular fluid and its main targets are granulosa and cumulus cells. The GDF-9 is fundamental for folliculogenesis and follicular development, by its action in recruiting and differentiating the somatic cells which will form the follicles, in addition to its mitogenic effect on the granulose and adjacent cumulus cells (Juengel, J. L., Hudson, N. L., Whiting, L. and McNatty, K. P. Effects of immunization against bone morphogenetic protein 15 and growth differentiation factor 9 on ovulation rate, fertilization, and pregnancy in ewes. Biol Reprod, 70, 557-561. 2004).

The BMP-15 and GDF-9 genes, as well as other members of the TGF-β family, are transcribed as pre-proteins composed by a peptide sign, a large pro-peptide and a mature region. After the removal of the peptide sign, the pro-peptide undergoes cleavage that separates it from the mature bioactive region. Recently it was demonstrated that just as other members of the TGF-β family, the dimerization of the pro-peptides is necessary for cleavage to occur and for hormones to be secreted. The proteins GDF-9 and BMP-15, just as other members of the TGF-β family, have a knot tertiary structure comprising 6 conserved cysteines, which form 3 sulphate bridges amongst themselves (Vitt et al., 2005). Evidence has shown that GDF-9 and BMP-15 mutually form homodimers and heterodimers.

The high fertility punctual mutation found in the GDF-9 gene was correlated to the increase in ovulation rate in ewes of the European strains Cambridge and Belclare when in heterozygosis and to infertility when in homozygosis. The homozygote individuals are sterile due to a deficiency in the development of primary follicles (Hanrahan et al., 2004). The mutation disclosed herein and on which the method of the invention is based, is situated on the 27^{th} codon of the mature peptide and causes a substitution of a phenylalanine for a cysteine. This alteration leads to the alteration of the hormonal function of the peptide encoded thereby, because the swapped phenylalanine is conserved in this position in various animals, which shows its importance in the protein structure. In fact, in the modeling of the mature peptide in its dimeric shaping, the phenylalanine is in a key position for interaction of GDF-9 sub-units.

There are other works in literature showing mutations in the GDF-9 gene and its implications for fertility. For example, document WO 03102199 presents mutations in the GDF- and GDF-9B (BMP-15) genes which alter the mammalian ovarian function and the ovulation rate. The invention has application both in the increase of ovulation rate or in the induction of sterility, since one of the mutations disclosed causes inactivation in GDF-9 gene when in homozygosis. The aforementioned document also reveals the sequence of the DNA and the polypeptide, resulting from the mutation occurred in GDF-9 gene.

The present invention discloses new mutations identified in the DNA sequence in the GDF-9 gene, different to those disclosed in document WO 03102199, which also alter the ovulation rate in mammals. Additionally, the method described in this invention enables a more accurate identification of prolific individuals, being of major importance for the selection and genetic enhancement of animals, besides assisting in the reproduction management of the flock.

Another possibility and the use of the mutated sequence for the production of transgenic animals with greater prolificacy. Azevedo (2007) (Azevedo, V. Aplicações da biotecnologia na área animal. Accessible at http://www.zoonews.com.br/noticias2/noticia. php?idnoticia=42826) highlights that since enhancement programs are conventional, by means of selection, rather slow and problematic, the gene transfer methods are highly attractive. In general, mammals are not high prolificacy animals, and even though some economically important species, such as pigs, present this characteristic, it is always useful to be able to control the ovulation rate of the flock. Transgenesis in animals has been more used in terms of modifications for resistance to diseases, to express changes in textile fibers, to alter the composition of milk, etc. Some transgenic animals have been developed with different utilities.

### SUMMARY OF THE INVENTION

The present invention refers to the identification of superior prolificacy animals, by means of precocious diagnosis of the presence of a genetic mutation.

In a preferred embodiment, the invention refers to a method of precocious and highly accurate identification of high prolificacy animals, based on a novel mutation in the GDF-9 gene using the technique of polymerase chain reaction. Said method is characterized by the steps of extracting the DNA from animals under study; amplification, characterization of the amplified patterns and identification of superior animals.

A second embodiment of the invention refers to a mutated DNA molecule, for use in the construction of a synthetic gene and its uses in obtaining transgenic animals for high prolificacy.

The third embodiment of the invention refers to the polypeptide encoded by the mutated gene and its uses in manipulating the mammalian ovarian functions.

Another embodiment of the invention refers to a kit for identifying high-prolificacy animals, composed of 2 suitable initiators, common reagents for PCR reaction and manual with instructions for identifying the results.

### BRIEF DESCRIPTION OF THE DRAWINGS

FIG. 1. Sequence of DNA nucleotides in the GDF-9 gene and peptidic sequence of the protein encoded thereby. The mutations described are indicated, with the new codon created and the aminoacids relating to the new codon, in the corresponding positions.

FIG. 2. Chromatograms obtained from the sequencing of mutant (Mut.) and wild-type (Selv.) DNAs for the mutations found. The arrows indicate the mutations observed.

FIG. 3. Alignment of the sequence of aminoacids of mature peptide GDF-9 for various species of mammals. The boxes indicate the cysteines that form sulphate bridges amongst themselves, forming a cysteine “knot”. The dashed box indicates the mutation found and the arrow indicates the substitution of aminoacids (phenylalanine for cysteine) located seven aminoacids before the first cysteine “knot”.

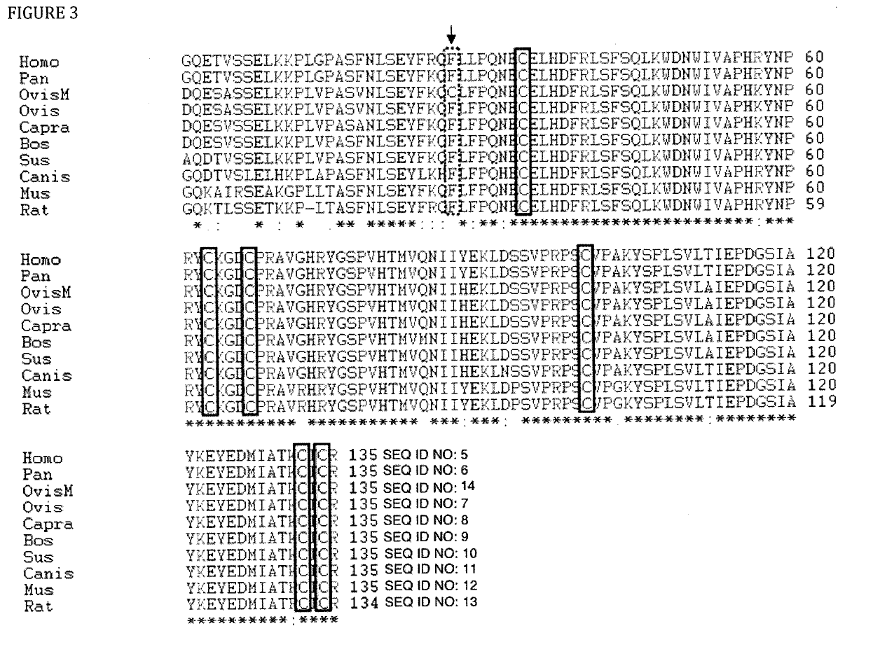

### DETAILED DESCRIPTION OF THE INVENTION

Punctual mutations in the GDF-9 gene were correlated to the increase in ovulation rate in ewes of European strains when in heterozygosis and to infertility when in homozygosis, described in patent document WO 03/102199.

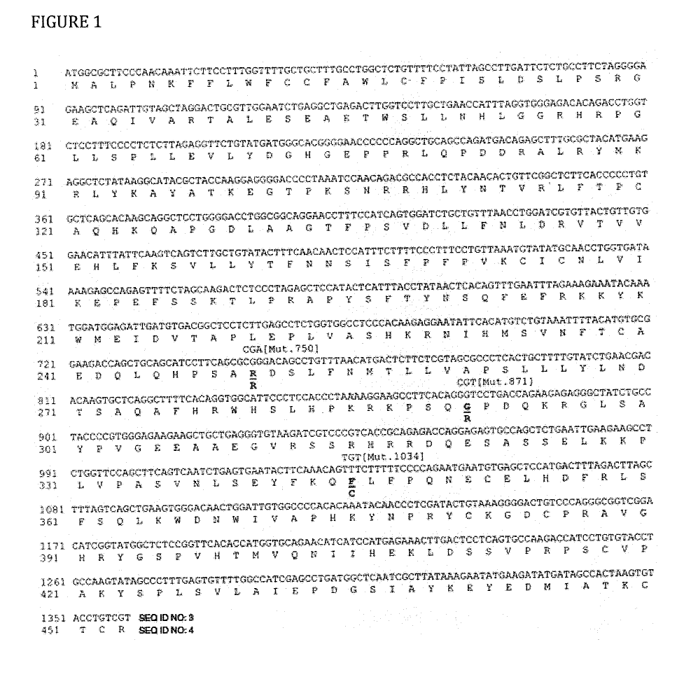

This invention describes a novel mutation in the GDF-9 gene, correlated to the increase in mammal prolificacy. Three mutation points are presented in different positions to those described in document W003/102199, which are shown in FIG. 1.

In document WO 03/102199, only one of the eight mutations described for GDF-9 has a positive effect on the ovulation rate, when in heterozygosis, leading to sterility when in homozygosis. Three novel mutations are described in the present invention, two of which are found in the pro-peptide, one does not alter the aminoacid of the mutated codon, and one causes an alteration in the aminoacid. The third mutation identified is in the region of the mature peptide, situated in the 27^{th} codon, which causes the substitution of the aminoacid phenylalanine for cysteine. Since this mutation is found only seven aminoacids away from the first cysteine of the “Knot”, this inserted cysteine establishes spurious sulphate bridges with some of the other cysteines of the Knot, altering the functional structure of the hormone. This fact is related to the formation of a new allele in the GDF-9 gene, which causes an increase in the ovulation rate.

After confirming the effect expected by this mutation on animal prolificacy, a method of predicting this characteristic was developed, by identifying animals carrying the mutation identified.

Accordingly, initiator oligonucleotides were designed based on the sequence deposited at the GenBank (AF078545), GDF-9 sense 5′-GGA GAA AAG GGA CAG AAG C-3′; anti-sense 5′-ACG ACA GGT ACA CTT AGT-3′. These initiators have proven to be effective in amplifying the sequence which contains the novel mutation in the GDF-9 gene, described in this document. All the reactions made amplified a fragment of 410 nucleotides in size, which when sequenced present mutation.

The invention therefore consists of a method for identifying animals with a high ovulation rate or high prolificacy.

The method proposed consists of the following steps:

(a) Extraction of samples of Genomic DNA from the blood, semen or other tissues of animals such as pilous follicle or any source of nucleated cells of the individual to be analyzed for the use of suitable initiators. Extraction can be performed using the “Salting out” protocol (Miller S. A., Dykes D. D. and Polesky H. F. A simple salting out procedure for extracting DNA from human nucleated cells, Nucleic Acids Research, vol 16(3) page 1215. 1988) or any procedure that allows access to the DNA of the cell nucleus.

(b) Amplification of the DNA relating to the GDF-9 gene, by polymerase chain reaction (PCR), using the initiators sense 5′-GGA GAA AAG GGA CAG AAG C-3 (SEQ. ID No. 1); anti-sense 5′-ACG ACA GGT ACA CTT AGT-3′(SEQ. ID No. 2),

(c) Digestion of the fragment produced by PCR described in (b), with appropriate restrictions enzymes, such as the TspRI restriction enzyme.

(d) Analysis of the digestion patterns of the fragment resulting from the digestion described in (c) by high-resolution electrophoresis, in capillary or in agarose gel, estimating the sizes of the products amplified by comparison with a known size standard.

(e) Identification of the genotypes(s) (genetic variant) associated to higher ovulation rate.

(f) Application of suitable statistical methods to interpret the results.

Known conditions and reagents are used in the DNA extraction step. For example, the extraction can be performed according to the procedures described by Miller et al. (1988) which, in short, consist of obtaining leucocytes from the total blood washed with a solution that promotes cell lysing; break the nuclear membrane of the leucocytes in the presence of a detergent such as SDS and a proteolytic enzyme such as Proteinase K; to remove the proteins from the solution by precipitation in the present of a high concentration of ions such as Na⁺Cl⁻; precipitate the DNA in the presence of an alcohol such as ethanol or isopropanol and solubilize it in an aqueous buffer solution preferably TE (Tris-EDTA).

The proteins may be alternatively excluded from the solution by organic solvents, such as phenol in the presence or not of chloroform and isoamilic alcohol. It is also possible to filter in a cellulose or silica column followed by elution of the DNA which adheres thereto.

The fragments are amplified using the PCR technique. In short, 20 to 200 ng of Genomic DNA is used; 0.1 to 0.3 mM of each dNTP; 1.5 to 2.0 mM MgCl₂; 20 mM TRIS pH 8.3; 50 mM KCl, 0.2 to 0.5 μM of each initiator and 1 to 1.5U of taq polymerase.

The amplifications are made in a thermocycler equipment which controls the temperatures and time desired. The thermocycling conditions consist of denaturation initially at 93-95° C. for two to five minutes, followed by 35 cycles of denaturation at 93-95° C. for 30 to 60 seconds, followed by annealing of the initiators between 58-64° C., for 30 to 60 seconds, and extension at 70-74° C. for 1 minute. A final extension between 70-74° C. for up to 10 minutes can be made.

After the amplifications, the products are digested with 0.2 to 1 unit of the TspRI enzyme at 65° C. for 18 to 24 hours in the presence of the enzyme buffer (Tris-acetate 20 mM, potassium acetate 50 mM, magnesium acetate 10 mM, dithiotreitol 1 mM, bovine seric albumin 0.1 mg/ml). After digestion the products are analyzed in agarose gel at 2.5% and the patterns of the fragments from digestion are established in base pairs according to the comparison with a suitable molecular marker. The data is interpreted by software accompanying the equipment, furnishing a table with identification of the animal and its respective genotype. Alternatively, it is possible to use eletrophoresis systems by capillarity in sequencing apparatus with the genotyping made by SNPs identification systems, such as, for example, using the Kit SNaPshot™ (Applied Biosystems) or similar.

The invention is also embodied in the form of a kit for identifying prolific animals, containing the initiators selected, flasks with common reagents that are necessary for the PCR reaction, in their adjusted concentrations, and a manual with instructions for identifying and interpreting the results.

### EXAMPLES The invention will now be described in greater detail by means of the following examples, which should not be interpreted as limitative on the scope of the invention.

### Example 1

### Sequencing of Exon 2 in the GDF-9 Gene in the Naturalized Brazilian Strain Santa Inês

The objective of this task was to sequence the exon 2 in the GDF-9 gene, which includes the coding region of the mature peptide, in ewes of the naturalized Brazilian strain Santa Inês. The ewes analyzed were descendents of multiple births, being candidates for presenting alterations in the GDF −9 gene.

DNA was extracted from the leucocyte fraction of the blood of Santa Inês ewes by “Salting Out” (Miller et al., 1988). Based on the Genomic DNA, the fragments corresponding to exon 2 in the GDF-9 gene of 13 animals selected for their pedigree were amplified by PCR.

Initiator oligonucleotides were designed, based on the sequence deposited at the GenBank (AF078545), GDF-9 sense 5′-GGA GAA AAG GGA CAG AAG C-3′; anti-sense 5′-ACG ACA GGT ACA CTT AGT-3′.

The PCR reactions were made with an end volume of 25 μL, with 5 pmoles from each initiator, 100 μL from each dNTP, 2 mM from MgCl₂, 1.5U of taq polymerase (Invitrogen) and approximately 100 ηg of DNA. The DNA was denatured at 93° C. for 3 minutes in a thermocycler (PT-100 MJ Research) and amplified for 35 cycles (93° C. for 40 seconds, 56° C. for 40 seconds, 72° C. for 1 minute). The samples were applied in agarose gel at 1.5%.

The amplified fragments were purified with the kit “Geneclean II” (Bio 101 Systems) and sequenced (sequencer ABI 3700). The initiator oligonucleotides used in sequencing in the GDF-9 gene were: 5′-GGA GAA AAG GGA CAG AAG C-3′ and 5′-GAC CAG GAG AGT GTC AGC-3′. The resulting sequences were aligned and subsequently refined in suitable computer programs.

The fragment obtained was sequenced and its components in nucleotides (SEQ ID No: 3) and in aminoacids (SEQ ID No 4) are shown in FIG. 1.

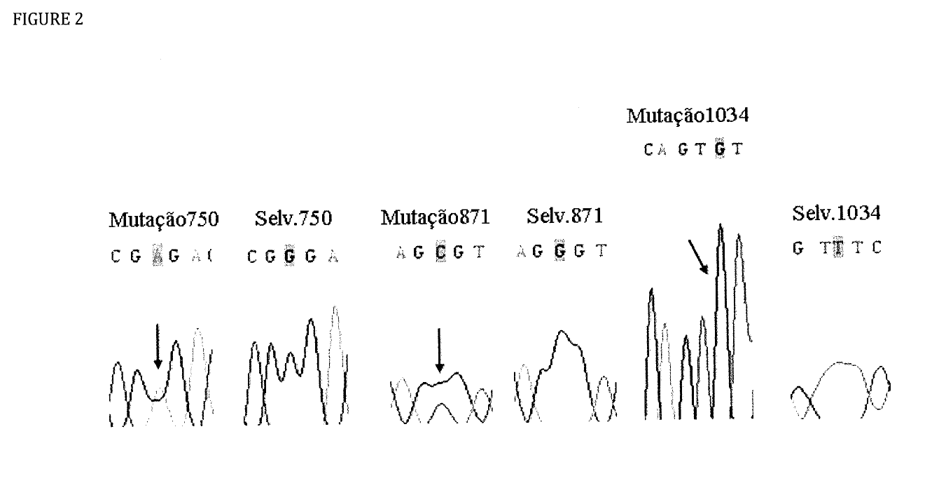

Three mutations (SNPs) were detected and are shown in Table 1 and FIG. 1, based on the chromatograms obtained (FIG. 2). Of these, one is silent and two are not conserved, and two of them are situated in the pre-peptide region. The main mutation is found in the region of the mature peptide and was observed in 10 of the 13 animals (Table 1). This mutation situated in the 27^{th} codon of the mature peptide leads to the substitution of the phenylalanine aminoacid to cysteine causing a disturbance in the knot of cysteines (SEQ ID No. 3).

TABLE 1
Types of mutations found in the animals
analyzed, their positions in the sequence analyzed, the
changes they cause in the synthesized aminoacids and the
degree of conservation thereof

| | Position in NT | | | |
|---|---|---|---|---|
| Type of | (relating to | Wild | Mutated | Conservation |
| Mutation | ATG) | codon | codon | level |
| T/G | 1034 (mature | TTT | TGT (cys) | Not |
| | peptide) | (phe) | | conserved |
| G/A | 750 (pre- | CGG | CGA (Arg) | Silent |
| | peptide) | (Arg) | | |
| G/C | 871 (pre- | GGT | CGT (Arg) | Not |
| | peptide) | (Gly) | | conserved |

Since the Santa Inês strain is formed by contributions from various breeds, the aim was to verify the existence of the mutation 1034 (Table 1) in other naturalized Brazilian breeds of sheep, including: Bergamácia, Somalis, Morada Nova, Rabo Largo and in European commercial breeds having no correlation with the Santa Inês strain, such as: Suffolk, Hampshire, Dorper, Ile-de-France and Corriedale.

Animals of the breeds specified above were genotyped by PCR-RFLP 517. The Genomic DNA was extracted from the blood leucocyte fraction of the animals of each strain and amplified by PCR using primers for the encoding region of the mature peptide.

After amplification of the fragment, a TspRI enzyme was used, being capable of distinguishing, by way of its products, between the wild allele and the mutant allele in the GDF-9 gene. Table 2 shows the results found.

TABLE 2
Number of animals studied and frequency of the
mutations in the different breeds tested

| | | Hetero- | | | | |
|---|---|---|---|---|---|---|
| Strain | Selv. | zygote | Homozygote | N | Freq. Wild/Mut | |
| Santa Inês | 149 | 72 | 7 | 228 | 0.8114 | 0.1886 |
| Somalis | 24 | 21 | 2 | 47 | 0.7340 | 0.2660 |
| Bergamácia | 24 | 20 | 0 | 44 | 0.7727 | 0.2272 |
| Morada Nova | 16 | 20 | 4 | 41 | 0.6585 | 0.3415 |
| Rabo Largo | 11 | 30 | 5 | 46 | 0.5652 | 0.4348 |
| Suffolk | 23 | 0 | 0 | 23 | 1.0000 | 0.0000 |
| Hampshire | 22 | 1 | 0 | 23 | 0.9792 | 0.0208 |
| Dorper | 23 | 1 | 0 | 24 | 0.9792 | 0.0208 |
| Ile-de- | 23 | 0 | 0 | 23 | 1.0000 | 0.0000 |
| France | | | | | | |
| Corriedale | 18 | 0 | 0 | 18 | 1.0000 | 0.0000 |

A mutation was found in the four breeds analyzed, but not in the New Zealand strain Corriedale, which was used as external control. The highest frequency of heterozygotes (0.66) was noted in the strain Rabo Largo, whereas the Morada Nova breed presented the highest frequency of homozygotes (0.15) for the mutant allele of GDF-9. The Santa Inês strain presented the highest frequency of animals without mutation (0.67). No homozygote animal was found among individuals of the Bergamácia strain, although the frequency of the homozygote allele was 0.2273.

In short, the mutation was found in the creole strains, but was not found in the other European breeds introduced subsequently to the formation of Santa Inês. This shows that the diagnostic mutation is of the Brazilian strains, and must have occurred in a common ancestor of one of these strains and subsequently transmitted by breeding to the other stocks. This also proves the difference between the mutation now described and that disclosed in document WO 03/102199.

### EXAMPLE 3

### Proof of the Relationship Between the Presence of the Mutation and the Increase in Ovulation Rate

To prove the relationship between the presence of the mutation 1034 characterized and the increase in the ovulation rate, a genotyping methodology was developed based on the PCR-RFLP technique with the amplification of the sequence of the mature peptide by PCR, followed by digestion with TspRI enzyme. This technique enabled genotyping and classification of the animals according to the presence of the mutated allele such as heterozygotes (He), homozygotes (Ho) and wild-type (Selv).

Subsequently, the effect of this mutation was evaluated on the ovulation rate, which was appraised by a laparoscopy examination measuring the quantity of corpora lutea in each ovary of the animals analyzed. The analysis comprised 35 ewes of the Santa Inês strain, gynecologically healthy and with a body condition over 3 (1-5). Of these, 15 had no mutation (Selv.), 16 were heterozygotes (HE) and four homozygotes for the mutation in GDF-9 gene (HO) (Table 3). The animals were synchronized with two doses of PGF2 (125 μg, intra-muscular) with an interval of nine days between applications. Detecting the estrus was performed with the assistance of a vasectomized ram and estrus was noted in all the ewes. The animals were submitted to laparoscopy eight days after the last application of PGF2, to identify and count the corpora lutea (CL), used to deduce the ovulation rate of the animals.

TABLE 3
Number of corpora lutea noted in animals of the
wild-type (Selv.), heterozygote (HE), and homozygote (HO)
genotype, with respect to mutation 1034.

| | | | # of Corpora lutea | | |
|---|---|---|---|---|---|
| | | Date of | (CL) | | Total |
| Animals | Genotype | Laparoscopy | Left Ovary | Right Ovary | CL |
| 017 | HO | Nov. 24 | 1 | 2 | 3 |
| 021 | HO | Nov. 23 | 1 | 1 | 2 |
| 04005 | HO | Nov. 23 | 1 | 1 | 2 |
| 04011 | HO | Nov. 24 | — | 2 | 2 |
| 018 | HE | Nov. 24 | 1 | — | 1 |
| 030 | HE | Nov. 23 | — | 1 | 1 |
| 038 | HE | Nov. 23 | 1 | — | 1 |
| 067 | HE | Nov. 23 | 1 | 1 | 2 |
| 637 | HE | Nov. 24 | — | 1 | 1 |
| 639 | HE | Nov. 23 | 1 | 1 | 2 |
| 730 | HE | Nov. 23 | 1 | 1 | 2 |
| 04004 | HE | Nov. 24 | 1 | — | 1 |
| 04119 | HE | Nov. 23 | — | 1 | 1 |
| 04056 | HE | Nov. 24 | 1 | — | 1 |
| 04101 | HE | Nov. 24 | 1 | — | 1 |
| 04112 | HE | Nov. 24 | 1 | — | 1 |
| 04125 | HE | Nov. 23 | — | 1 | 1 |
| 04126 | HE | Nov. 23 | 1 | — | 1 |
| 064 | HE | Nov. 23 | 1 | — | 1 |
| 05015 | HE | Nov. 24 | — | 1 | 1 |
| 032 | Selv. | Nov. 24 | 1 | — | 1 |
| 046 | Selv. | Nov. 23 | 1 | — | 1 |
| 04039 | Selv. | Nov. 23 | — | 1 | 1 |
| 04065 | Selv. | Nov. 24 | 1 | — | 1 |
| 540 | Selv. | Nov. 24 | — | 2 | 2 |
| 04103 | Selv. | Nov. 23 | — | 1 | 1 |
| 04102 | Selv | Nov. 24 | 1 | — | 1 |
| 04110 | Selv. | Nov. 23 | 1 | — | 1 |
| 04114 | Selv. | Nov. 23 | — | 1 | 1 |
| 04117 | Selv. | Nov. 24 | 1 | — | 1 |
| 04124 | Selv. | Nov. 24 | 1 | — | 1 |
| 04143 | Selv. | Nov. 23 | 1 | — | 1 |
| 04158 | Selv. | Nov. 23 | — | 1 | 1 |
| 04167 | Selv. | Nov. 24 | — | 1 | 1 |
| 05033 | Selv. | Nov. 24 | 1 | — | 1 |

For the comparison between groups with variable binomials, the Fisher's Exact Test was used, and for continual variables, the Kruskal-Wallis non-parametric test was used. The laparoscopic examination revealed that 100% (35/35) of the ewes presented at least one CL. In the inter-group comparison, homozygote ewes for the GDF-9 gene (HO) showed a higher percentage of multiple ovulations (75.0% double ovulation and 25% triple ovulation) when compared to the Selv. or HE (P<0.05) groups, with 6.7% and 18.8% of double ovulations respectively. A greater number of CL per ewe in the HO (2.25±0.25; average±standard error) group was also confirmed in relation to the WT (1.07±0.07) or HE (1.19±0.10) groups.

It is concluded that the mutation in the GDF-9 gene is related to the increase in the ovulation rate in ewes of the Santa Inês strain. This marker can be used as an important parameter for the selection of animals with a greater reproductive potential and, accordingly, obtain a substantial increment in the production of sheep for slaughter in Brazil.

1. Method of identifying high prolificacy genotypes in mammals, comprising:
(a) extraction of nucleic acid (genomic DNA) from the animals under investigation;
(b) amplifying a certain region of the genome from a pair of specific initiators, near the gene(s) related to fertility in animals;
(c) digesting the fragment obtained in (b) with restriction enzymes;
(d) analyzing the products amplified; and
(e) comparing the products amplified under investigation to determine their genotype;
(f) applying suitable statistical methods to interpret the results.

2. Method according to claim 1 wherein at least one sequence is located in a region of the genomic DNA related to the gene GDF-9 (“Growth and Differentiation Factor 9”).

3. Method according to claim 1 wherein the animals are selected from a group of mammals.

4. Method according to claim 2 wherein the animals are selected from a group of ewes.

5. Method according to claim 1 wherein the pair of initiators is selected from the group consisting of (i) SEQ ID No. 1 and SEQ ID No. 2.

6. Method according to claim 1 wherein said separation of step (c) is made by electrophoresis.

7. Method according to claim 9 wherein said electrophoresis is high resolution in agarose polymers.

8. Method according to claim 1 wherein said identification of the amplified products is by fluorescence.

9. Method according to claim 1, wherein said identification of amplified products is by radiation marking.

10. Method according to claim 1, wherein it is used in animal genetic enhancement programs.

11. Molecule of nucleic acid isolated from the mutated gene GDF-9, comprising a nucleotide sequence substantially identical to SEQ ID No: 3.

12. Polypeptide encoded by the mutated gene GDF-9, comprising mutation 1034.

13. Polypeptide according to claim 1, wherein the sequence is substantially identical to SEQ ID No: 3.

14. DNA construct comprising a suitable promoter, operationally bound to a polynucleotide having a sequence of nucleotides identical to SEQ ID No: 3, coding for a polypeptide having a sequence of aminoacids substantially identical to SEQ ID No 4, said polynucleotide being operationally bound to a transcription region.

15. Transformed cell comprising a nucleic acid coding for a polypeptide having a sequence of aminoacids substantially identical to SEQ ID No: 4, or that contains mutation 1034, having activity on mammalian ovarian functions.

16. Transgenic animal comprising a nucleic acid incorporated to its genome having a sequence of nucleotides substantially identical to SEQ ID No: 3.

17. Kit for identifying high prolificacy animals comprising the following components:
(a) set of marker initiators comprising at least one from among: (i) SEQ ID No. 1 and SEQ ID No. 2;
(b) reagents for performing PCR; and
(c) instructions for use in identifying animals under investigation.
